(19) United States
(12) Patent Application Publication
(10) Pub. No.: US 2019/0151225 A1
(43) Pub. Date: May 23, 2019

(54) Waterborne Composition, Transparent Film And Method Of Forming The Same For Covering Acne

(71) Applicant: Tair Jiuh Enterprise Co., Ltd., Tainan City (TW)

(72) Inventors: Po-Han Chen, Tainan City (TW); Ya-Yun Tsai, Tainan City (TW); Chia-Li Huang, Tainan City (TW); I-Ming Chen, Tainan City (TW); Tung-Liang Chen, Tainan City (TW)

(21) Appl. No.: 15/970,370

(22) Filed: May 3, 2018

(30) Foreign Application Priority Data
Nov. 22, 2017 (TW) 106140616

(51) Int. Cl. A61K 8/85; A61K 8/87; A61K 8/67; A61K 8/9783; A61K 8/73; A61K 8/34; A61Q 17/00; A61P 17/10

(52) U.S. Cl. CPC A61K 8/85; A61K 8/87; A61K 8/675; A61K 8/9783; A61K 2800/48; A61K 8/345; A61Q 17/00; A61P 17/10; A61K 8/73

(57) ABSTRACT

The present invention provides a waterborne composition for preventing contact between acnes and irritants, and the water based composition includes a polymer (A), a thickener (B), a functional additive (C) and water (D). The present invention further provides a transparent film and a method of forming the same for preventing contact between acnes and irritants.

### RELATED APPLICATIONS

This application claims priority to Taiwan Application Serial Number 106140616, filed Nov. 22, 2017, which is herein incorporated by reference.

### BACKGROUND

### Field of Invention

The present invention relates to a waterborne composition, a transparent film and a method of forming the transparent film for covering an acne. More particularly, the present invention relates to a waterborne composition including a specific polymer and a functional additive, a transparent film which is less irritating, conformal to a skin and long acting without formation of scraps, and a method of forming the transparent film.

### Description of Related Art

An acne, which is usually called as a pimple, is a common skin problem. Generally, the acne is a skin disease caused by clogged sebaceous gland, and often occurs on a face, a neck, a front chest or a back chest where a lot of sebaceous glands are distributed.

In general, cosmetics such as a foundation or a blush powder should not be coated on the acne to prevent irritating and worsening the acne, while great inconvenience occurs. In addition, with the worsening air quality, suspended particles in air are also likely to irritate the acne and cause a negative effect on the acne.

There are some common products or methods can prevent the contact between the acne and the irritants. One of the common products is an acne patch or a wound dressing. However, both the acne patch and the wound dressing include a supported substrate to secure the products on the acne. When the products are used, an obvious trace and protrusion unfavorable to makeup are formed.

Another common method is to apply a film-forming gel composition which is applicable to the wound. The film-forming gel composition can prevent the contact between the acne and the irritants, however, problems such formation of scraps due to the over-thick film, significant irritating sensation (due to containing the organic solvent such as ethanol for drying the composition faster), unsatisfied appearance due to film whitening may occur.

In view of the problems described above, a waterborne composition, a transparent film and a method of forming the same are required. The waterborne composition can form the transparent film in a shorter time, and the transparent film is thin, conformal to the skin, less irritating and the scraps will not be formed after the film is rubbed. Furthermore, the supported substrate is not required in the transparent film.

### SUMMARY

One aspect of the invention provides a waterborne composition for covering the acne. The waterborne composition includes particular amounts of a specific polymer and a specific functional additive in water, so as to prevent other outer substances from contacting with the acne and treat the acne in a shorter time.

According to the aspect, a waterborne composition is provided. The waterborne composition includes a polymer (A), a thickener (B), a functional additive (C) and water (D). The polymer (A) includes polyester-5, polyurethane-32 and/or polyurethane-48. The functional additive (C) includes an onion extract, vitamin B3, vitamin B5 or a combination thereof. Based on an amount of the polymer (A) as 100 parts by weight, an amount of the thickener (B) is 2 parts by weight to 20 parts by weight, an amount of the functional additive (C) is 15 parts by weight to 150 parts by weight, and an amount of the water (D) is 380 parts by weight to 4700 parts by weight.

According to one embodiment of the present invention, the thickener (B) is xanthan gum.

According to one embodiment of the present invention, a viscosity of the waterborne composition is smaller than 2000 cps.

According to one embodiment of the present invention, the amount of the water (D) is 80 weight percent (wt. %) to 94 wt. % of an amount of the waterborne composition.

According to one embodiment of the present invention, the waterborne composition further includes an alcohol (E), and based on the amount of the polymer (A) as 100 parts by weigh, an amount of the alcohol (E) is 3 parts by weight to 30 parts by weight.

According to one embodiment of the present invention, the alcohol (E) includes phenoxyethanol, caprylyl glycol, butylene glycol, pentylene glycol, hexylene glycol or a combination thereof.

According to one embodiment of the present invention, the waterborne composition excludes a lower monoalcohol organic solvent.

The other aspect of the present invention is to provide a method of forming a transparent film for covering the acne. The method includes performing a coating step for applying the waterborne composition described above on acne on a surface, thereby forming a coating layer on the surface. An amount of the waterborne composition used in the coating step is 1 mg/cm² to 100 mg/cm² of the surface. The method further includes naturally drying the coating layer for 40 seconds to 1.5 minutes, thereby forming the transparent isolation film.

Another aspect of the present invention is to provide a transparent film for covering acne. The transparent film is formed by the above method. And the transparent film is adhered on acne on a skin for at least 4 hours.

According to one embodiment of the present invention, the transparent film is a single-layered film without a supported substrate.

The contact between the acne and the irritants (such as a variety of cosmetics or the suspended particles in air) is prevented by applying the waterborne composition, the transparent film and the method of forming the same, so as to prevent repeated inflammatory episodes of the acne.

### DETAILED DESCRIPTION

A purpose of the present invention is directed to providing a waterborne composition for covering acne. A great part of the composition is water, and the composition further includes a specific polymer and a specific functional additive. A transparent film may be directly formed on a skin (or acne on the skin) by the waterborne composition, thereby preventing the contact between the acne and the irritants. Anti-inflammatory and soothing effects are provided by the transparent film, and the acne may be healed in a shorter time. Furthermore, the waterborne composition can be dried rapidly, and the rapid drying rate is beneficial for formation of the transparent film. Moreover, the transparent film is colorless and has good conformability to the skin, and thus the transparent film will not influence a color of cosmetics on the skin, and scraps caused by an over-thick film are prevented.

All components of the waterborne composition described herein belong to hydrophilic substances, and a hydrophilic polymer is particularly included in the composition. A macrostructure and hydrophilicity of the polymer inhibit the polymer from being absorbed by the skin, and thus the waterborne composition is less irritating to the skin. In addition, a hydrophilic functional additive is secured in a specific position by the film formed from the polymer, thereby soothing or treating the acne by continually applying the hydrophilic functional additive onto the acne.

The irritants described herein may be referred to substances that are likely to worsen inflammation of the acne, such as the cosmetics, a skin-care product, a suspended particle in air, and the like.

The waterborne composition of the present invention includes a polymer (A), a thickener (B), a functional additive (C) and water (D). Based on an amount of the polymer (A) as 100 parts by weight, an amount of the thickener (B) is 2 parts by weight to 20 parts by weight, an amount of the functional additive (C) is 15 parts by weight to 150 parts by weight, and an amount of the water (D) is 380 parts by weight to 4700 parts by weight.

In one embodiment, the polymer (A) includes polyester-5, polyurethane-32 and/or polyurethane-48. Specific polymers and the specific amount of the polymers are selected, such that the transparent film formed from the waterborne composition may have adequate softness to provide sufficient conformability to the skin. Moreover, other benefits of using the specific polymer (A) include a reduced tight-feeling or irritating-sensation of the skin.

When the polymer (A) is replaced by other polymers such as poly vinyl alcohol (PVA), sodium polyacrylate or nitrocellulose, problems like poor solubility of the polymer, formation of the tough film having insufficient conformability, and whitening of the film may occur. In addition, when the amount of the polymer (A) is not within the disclosed range of the present invention, the transparent film cannot be formed if the amount of the polymer (A) is too less (fails to form a complete film to cover the acne). On the other hand, scraps are easily formed from the transparent film after the film is rubbed if the amount of the polymer (A) is greater than the upper limit. For example, the transparent is rubbed when coating the cosmetics (e.g. a foundation) onto the skin.

In one embodiment, the thickener (B) is xanthan gum, which is used to control the viscosity of the waterborne composition of the present invention to be equal to or smaller than 2000 cps. When the viscosity of the waterborne composition is greater than 2000 cps, several disadvantages may occur, such as formation of an over-thick film, significant tight-feeling, formation of the scraps after the transparent film is rubbed. Furthermore, the xanthan gum is a relative stable thickener among common thickeners, since the xanthan gum is less likely to be influenced by the pH value and precipitation is less likely to be formed. Therefore, the xanthan gum is advantageous for the formation of the transparent film. It is noted that carbomer is excluded from the thickener of the present invention since the carbomer has poor film-forming property, and the carbomer is more likely to be influenced by the pH value and not resistant to ions, causing formation of the precipitation.

In one embodiment, the functional additive (C) is an onion extract, vitamin B3, vitamin B5 or a combination thereof. There are a variety of anti-inflammatory or anti-bacterial components which are known for treating the acne, for example, chamomile extract or epilobium angustifolium extract. However, when the other additives but not the functional additive (C) of the present invention are included in the water-base composition, for example, the chamomile extract or the epilobium angustifolium extract described above, problems such as formation of the precipitation or yellowing of the waterborne composition usually occur, and the transparent film cannot be formed.

In one embodiment, the water (D) may be deionized water. In other embodiments, the waterborne composition may further include an alcohol (E). Generally, phenoxyethanol, caprylyl glycol, butylene glycol, pentylene glycol, hexylene glycol or a combination thereof may be used as the alcohol (E) for a better preservation of the waterborne composition, and the alcohol (E) may cause less irritating sensation of the waterborne composition. In one example, based on the amount of the polymer (A) as 100 parts by weigh, an amount of the alcohol (E) is 3 parts by weight to 30 parts by weight. It is noted that most of the examples of the alcohol (E) disclosed here belong to a glycol compound. However, when the amount of the glycol compound (e.g. used as a solubilizer) is greater the upper limit of the present invention, a hydrogen bond is likely to be formed between the glycol compound and the waterborne composition, leading to reduction of a drying rate of the waterborne composition (i.e. a rate for forming the transparent film). In addition, the present invention selects specific alcohol (E) for the waterborne composition. When other types of the alcohols are applied in the waterborne composition, the scraps are likely to be formed after the transparent film of the waterborne composition is rubbed.

In a preferable embodiment, the amount of the water (D) is 80 weight percent (wt. %) to 94 wt. % of a total amount of the waterborne composition. It is noted that a lower monoalcohol organic solvent is excluded from the waterborne composition of the present invention due to an irritating property of the organic solvent. For example, the lower monoalcohol organic solvents having 1 to 4 carbon atoms are excluded.

The other purpose of the present invention is directed to providing a method of forming a transparent film for covering acne. The method includes performing a coating step for applying the waterborne compositing described above on acne on a surface, thereby forming a coating layer on the surface. An amount of the waterborne composition used in the coating step is 1 mg/cm² to 100 mg/cm² of the surface. The method further includes naturally drying the coating layer for 40 seconds to 1.5 minutes, thereby forming the transparent film.

In one example, the aforementioned surface may be a human skin, and the coating step may be performed by, for example, directly disposing the waterborne composition on the human skin and uniformly smearing the waterborne composition by a cotton swab or other medical materials.

Another purpose of the present invention is directed to providing a transparent film for covering acne. The transparent film is formed by the above method. The transparent film is adhered on acne on the skin for at least 4 hours. In other words, the transparent film of the present invention can be adhered to the skin surface in a longer time, so as to continually release the functional additive of the transparent film to the surface of the acne for a long-term treatment.

In one embodiment, the transparent film is a single-layered film without a supported substrate. The supported substrate may be, for example, fabrics, cloths, papers, and the like. In other words, the transparent film is independently formed by the aforementioned method, and the transparent film is able to cover the acne. Use of the supported substrate leads to insufficient conformability to the skin surface, and the color of the supported substrate influences the color and the uniformity of the cosmetics on the skin.

Examples and Comparative examples are provided as follows to describe the specific implementation of the present invention.

### EXAMPLE 1

First, 100 parts by weight of the polyester-5 (product number: AQ55S, provided by Easchem Co., Ltd.) was dissolved in 4700 parts by weight of water, and the solution was continually stirred for 3 minutes in a water bath at 70° C. to 80° C. Then, 20 parts by weight of xanthan gum (product number: NovaXan 200T) was added into the solution during a natural cooling process, followed by stirring the solution for 10 minutes at a stirring rate of 270 rpm to 1010 rpm. Thereafter, 150 parts by weight of an onion extract (product number: 4358 100G ONION EXTRA GNBN, provided by Kosfarm corporation) was added when the temperature of the solution decreased to 45° C. Next, 30 parts by weight of phenoxyethanol/caprylyl glycol was added and stirred for 7 minutes at the stirring rate of 660 rpm, so as to form a waterborne composition of Example 1. An evaluation result of Example 1 is shown in Table 1.

### EXAMPLE 2

First, 82.57 parts by weigh of the polyurethane-32 (product number: Baycusan C1003) and 17.43 parts by weigh of the polyurethane-48 (product number: Baycusan C1008) were dissolved in 941.78 parts by weight of water, and the solution was continually stirred for 10 minutes at the stirring rate of 400 rpm. Then, 4.34 parts by weight of xanthan gum was added into the solution, followed by stirring the solution for 30 minutes at a stirring rate of 900 rpm. Thereafter, 32.55 parts by weight of the onion extract was added and stirred for 2 minutes at the stirring rate of 900 rpm. Next, 6.5 parts by weight of phenoxyethanol/caprylyl glycol was added and stirred for 10 minutes at the stirring rate of 900 rpm, so as to form a waterborne composition of Example 2. An evaluation result of Example 2 is shown in Table 1.

### EXAMPLES 3 TO 5 AND COMPARATIVE EXAMPLES 1 TO 6

Examples 3 to 5 and Comparative examples 1 to 6 were performed in the same way as the Example 1 or Example 2, in which the method of Example 1 was applied for the examples used polyester, and the method of Example 2 was applied for the examples used polyurethane. The difference was that Examples 3 to 5 and Comparative examples 1 to 6 adjusted the species or the amounts of the composition. Evaluation results of Examples 3 to 5 and Comparative examples 1 to 6 are referred to Table 1.

### Evaluation Result

### 1. Transparency

The transparency of the present invention is evaluated by a visual color of the transparent film. The transparent film is formed by coating the waterborne composition of Examples and Comparative examples on the human skin, and naturally drying the composition. The specific evaluation standards are shown as follows:
⊚: The transparent film cannot be observed.
◯: Some traces of the transparent film are observed.
×: The transparent film is clearly observed (e.g. a whitened film).

### 2. Rate of Forming the Transparent Film

The rate of forming the transparent film of the present invention is evaluated by the time of forming the transparent film. The transparent film is formed by coating the waterborne composition of Examples and Comparative examples onto the human skin and drying at a room temperature (e.g. 25° C.). The dryness of the film is defined by a non-sticky feel when the film is touched by hands. The evaluation standards are shown as follows:
⊚: The transparent film is formed in 40 seconds to 1 minute.
◯: The the transparent film is formed in more than 1 minute to 1.5 minutes.
×: The transparent film is formed in more than 1.5 minutes.

### 3. Amount of the Composition for Forming the Transparent Film

An amount of the composition for forming the transparent film is corresponding to a lower limit of the amount of the waterborne composition per unit area of the human skin for forming the transparent film. The evaluation standards are shown as follows:
⊚: An amount of the waterborne composition for forming the transparent film is 1 mg/cm² to 20mg/cm² of the human skin.
◯: An amount of the waterborne composition for forming the transparent film is 20 mg/cm² to 100 mg/cm² of the human skin.
×:An amount of the waterborne composition for forming the transparent film is greater than 100mg/cm² of the human skin.

### 4. Formation of Scraps After the Transparent Film is Rubbed

The evaluation is performed by rubbing the transparent film of the waterborne composition of Examples and Comparative examples, and observing if there are any white scraps formed. The evaluation standards are shown as follows:
⊚: No scraps are formed.
×: Some scraps are formed.

### 5. Irritating Property

The irritating property of the waterborne composition is evaluated by a sensory test. The waterborne composition of Examples and Comparative examples are coated on the acne of the skin, followed by a judgment of irritating sensation such as stabbing pain, burning pain or redness by a subject. The evaluation standards are shown as follows:
⊚: No irritating sensation.
×: Feeling irritated.

### 6. Anti-Inflammatory Effect on the Acne

The anti-inflammatory effect on the acne of the present invention is evaluated by coating the waterborne composition of Examples and Comparative examples on the acne of the skin, followed by measuring the time that the inflammation is improved. The evaluation standards are shown as follows:
⊚: The inflammation is improved in one day.
◯: The inflammation is improved in seven days.
×: The inflammation is improved in 14 days or not improved.

As shown in Examples 1 to 5 in Table 1, the waterborne composition of the present invention has good transparency, a rapid rate of forming the transparent film and reduced amount of the composition for forming the film. Further, the waterborne composition of the present invention does not form the scraps, does not cause irritating sensation to the skin, and has good anti-inflammatory effect. On the other hand, according to Comparative examples 1 to 6, when the specific polymer (A), the thickener (B) and/or the functional additive (C) disclosed in the present invention is not used, the waterborne composition has problems such as poor transparency, slow rate of forming the transparent film and formation of the scraps. In addition, when the amount of the water (D) is greater than the upper limit of the present invention, the film-forming property of the composition is insufficient, causing poor ant-inflammatory effect of the transparent film. Due to incomplete formation of the transparent film, the transparent film in unable to cover the acne on the skin.

In addition, the waterborne composition of the present invention forms the transparent film that can be adhered to the skin for at least 4 hours, so as to continually cover the acne and provide the anti-inflammatory effects in some embodiments.

The waterborne composition, the transparent film and the method of forming the same for covering acne of the present invention provide good transparency, a rapid rate of forming the transparent film, a reduced amount of the composition for forming the film, less irritating sensation and good anti-inflammatory effect without the formation of the scraps. Moreover, the waterborne composition has good conformability to the skin. Therefore, the small wound (e.g. the acne) on the skin may be separated from the outer irritants by applying the waterborne composition of the present invention without influencing the makeup effect.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

TABLE 1

| | | Example | | | | | Comparative example | | | | | |
|---|---|---|---|---|---|---|---|---|---|---|---|---|
| | | 1 | 2 | 3 | 4 | 5 | 1 | 2 | 3 | 4 | 5 | 6 |
| polymer (A) | A-1 | 100 | — | 100 | 100 | — | 100 | 100 | — | — | — | — |
| | A-2 | — | 82.57 | — | — | 82.57 | — | — | 75 | 75 | — | — |
| | A-3 | — | 17.43 | — | — | 17.43 | — | — | 25 | 25 | — | — |
| | A′-1 | — | — | — | — | — | — | — | — | — | 100 | — |
| | A′-2 | — | — | — | — | — | — | — | — | — | — | 100 |
| thickener (B) | B-1 | 20 | 4.34 | — | 20 | 4.34 | 20 | 20 | — | 2 | 20 | 20 |
| | B′-1 | — | — | — | — | — | — | — | 2 | — | — | — |
| functional | C-1 | 150 | 32.55 | — | 50 | 32.55 | — | — | 10 | 15 | — | 150 |
| additive (C) | C-2 | — | — | 100 | 50 | 10.85 | — | — | — | — | — | — |
| | C-3 | — | — | 50 | 50 | 10.85 | — | — | — | — | — | — |
| | C′-1 | — | — | — | — | — | 150 | — | — | — | 150 | — |
| | C′-2 | — | — | — | — | — | — | 150 | — | — | — | — |
| water (D) | D-1 | 4700 | 941.78 | 4700 | 4700 | 941.78 | 4700 | 200 | 380 | 380 | 5000 | 4700 |
| perspective (E) | E-1 | 30 | 6.5 | 30 | 30 | 6.5 | 40 | 30 | 3 | — | 30 | 30 |
| solubilizer (F) | F-1 | — | — | — | — | — | — | — | — | 32.5 | — | — |
| physical | viscosity (cps) | 1750 | 1950 | 1780 | 1700 | 2000 | 1750 | — | — | 1650 | — | — |
| property | | | | | | | | | | | | |
| Evaluation | transparency | ⊚ | ◯ | ⊚ | ⊚ | ◯ | X | X | X | ◯ | X | ◯ |
| result | film-forming | ⊚ | ◯ | ⊚ | ⊚ | ◯ | ◯ | — | X | X | X | X |
| | rate | | | | | | | | | | | |
| | amount of the | ⊚ | ⊚ | ⊚ | ⊚ | ⊚ | ◯ | — | ◯ | ◯ | ◯ | ◯ |
| | composition | | | | | | | | | | | |
| | scraps | ⊚ | ⊚ | ⊚ | ⊚ | ⊚ | ◯ | — | X | X | X | X |
| | irritating | ⊚ | ⊚ | ⊚ | ⊚ | ⊚ | ⊚ | — | ⊚ | ⊚ | ⊚ | ⊚ |
| | property | | | | | | | | | | | |
| | anti- | ⊚ | ◯ | ◯ | ⊚ | ⊚ | ◯ | — | ◯ | ⊚ | X | ⊚ |
| | inflammatory | | | | | | | | | | | |
| | effect | | | | | | | | | | | |

| | | |
|---|---|---|
| A-1 | polyester-5 | product number: AQ55S, provided by |
| | | Easchem Co., Ltd. |
| A-2 | polyurethane-32 | product number: Baycusan C1003, provided by |
| | | Tri-Matix industrial Co., Ltd. |
| A-3 | polyurethane-48 | product number: Baycusan C1008, provided by |
| | | Tri-Matix industrial Co., Ltd. |
| A′-1 | poly vinyl alcohol | product number: BP-17, provided by First |
| | | Chemical Co., Ltd. |
| A′-2 | sodium | product number: 2E0037, provided by First |
| | polyacrylate | Chemical Co., Ltd |
| B-1 | xanthan gum | product number: NovaXan 200T, provided by |
| | | Honor Chem Co., Ltd. |
| B′-1 | carbomer | product number: 2EG060, provided by First |
| | | Chemical Co., Ltd |
| C-1 | onion extract | product number: 4358 100G |
| | | ONION EXTRA GNBN, |
| | | provided by Kosfarm corporation |
| C-2 | vitamin B3 | product number: Niacinamide, provided by |
| | | Essence plus Co., Ltd. |
| C-3 | vitamin B5 | product number: Panthenol, provided by |
| | | Essence plus Co., Ltd. |
| C′-1 | epilobium | product number: CANADIAN |
| | angustifolium | WILLOWERB(cw-5c), provided by |
| | extract | Top Rhyme International Co, Ltd. |
| C′-2 | chamomile | product number: 43600 CHAMOMILE-ECO, |
| | extract | provided by Kosfarm corporation |
| D-1 | deionized water | — |
| E-1 | phenoxyethanol/ | product number: Verstatil PC, provided by |
| | caprylyl glycol | Honor Chem Co., Ltd. |
| F-1 | propylene glycol | product number: ZEMA, provided by Taiwan |
| | | High and Better Corp. |

What is claimed is:

1. A waterborne composition for covering acne, the waterborne composition comprising:
a polymer (A), comprising polyester-5, polyurethane-32 and/or polyurethane-48;
a thickener (B);
a functional additive (C), comprising an onion extract, vitamin B3, vitamin B5 or a combination thereof; and
water (D),
wherein based on an amount of the polymer (A) as 100 parts by weight, an amount of the thickener (B) is 2 parts by weight to 20 parts by weight, an amount of the functional additive (C) is 15 parts by weight to 150 parts by weight, and an amount of the water (D) is 380 parts by weight to 4700 parts by weight.

2. The waterborne composition of claim 1, wherein the thickener (B) is xanthan gum.

3. The waterborne composition of claim 1, wherein a viscosity of the waterborne composition is smaller than 2000 cps.

4. The waterborne composition of claim 1, wherein the amount of the water (D) is 80 weight percent (wt. %) to 94 wt. % of a total amount of the waterborne composition.

5. The waterborne composition of claim 1, further comprising an alcohol (E), and based on the amount of the polymer (A) as 100 parts by weigh, an amount of the alcohol (E) is 3 parts by weight to 30 parts by weight.

6. The waterborne composition of claim 5, wherein the alcohol (E) includes phenoxyethanol, caprylyl glycol, butylene glycol, pentylene glycol, hexylene glycol or a combination thereof.

7. The waterborne composition of claim 1, wherein the waterborne composition excludes a lower monoalcohol organic solvent.

8. A method of forming a transparent film for covering acne, the method comprising:
performing a coating step for applying a waterborne composition of claim 1 on an acne on a surface, thereby forming a coating layer on the acne, wherein an amount of the waterborne composition is 1 mg/cm² to 100 mg/cm² of the surface; and
naturally drying the coating layer for 40 seconds to 1.5 minutes, thereby forming the transparent film.

9. A transparent film for covering acne formed by the method of claim 8, wherein the transparent film is adhered on acne on a skin for at least 4 hours.

10. The transparent film of claim 9, wherein the transparent film is a single-layered film without a supported substrate.
